(19) United States
(12) Patent Application Publication
(10) Pub. No.: US 2014/0039388 A1
(43) Pub. Date: Feb. 6, 2014

(54) MEDICAL DEVICE FOR INTRACORPOREAL USE

(71) Applicant: BIOTRONIK AG, Buelach (CH)

(72) Inventor: Christian Blohm, Nuernberg (DE)

(73) Assignee: BIOTRONIK AG, Buelach (CH)

(21) Appl. No.: 13/948,932

(22) Filed: Jul. 23, 2013

Related U.S. Application Data
(60) Provisional application No. 61/677,490, filed on Jul. 31, 2012.

(51) Int. Cl. A61F 2/82; A61N 1/362; A61M 25/10; A61N 1/39

(52) U.S. Cl. CPC A61F 2/82; A61N 1/39; A61N 1/362; A61M 25/10
USPC 604/96.01; 623/23.7; 607/5; 607/9

(57) ABSTRACT

A medical device for intracorporeal use formed at least in part from aerographite.

### CROSS REFERENCE TO RELATED APPLICATIONS

This invention claims benefit of priority to U.S. provisional patent application Ser. No. 61/677,490 filed Jul. 31, 2012; the contents of which are herein incorporated by reference in their entirety

### TECHNICAL FIELD

The present invention relates to a medical device for intracorporeal use.

### BACKGROUND

The use of implants or medical devices for intracorporeal diagnostic purposes or for treatment represents a very essential aspect of modern medical engineering. Medical devices developed for these purposes have to satisfy a very wide range of demands. Apart from the actual intended purpose of the devices, for example the material selection in the case of implants is of considerable importance for long-lasting compatibility,

The term “implant” is to be understood generally to mean any medical device formed from one or more materials, which is intentionally introduced into the body and is covered either in part or completely by an epithelial surface. Implants can be subdivided in terms of the period of use into temporary implants and permanent implants. Temporary implants remain in the body for a limited period of time. Permanent implants are intended to remain permanently in the body. In the case of implants, a distinction can also be made between prostheses and artificial organs. A prosthesis is a medical device that replaces bodily limbs, organs or tissue, whereas an artificial organ is understood to be a medical device that partially or completely replaces the function of a bodily organ. The aforementioned definitions for example include implants, such as orthopedic or osteosynthetic implants, cardiac pacemakers and defibrillators and vascular implants.

In particular, the implantation of stents has become established as one of the most effective therapeutic measures in the treatment of vascular diseases. Stents are used to perform a supporting function in a patient's hollow organs. For this purpose, stents of conventional design have a filigree supporting structure formed from metal struts, which is initially provided in a compressed form for introduction into the body and is expanded at the site of application. One of the main fields of application of such stents is the permanent or temporary widening and maintained opening of vascular constrictions, in particular of constrictions (stenoses) of the coronary vessels. In addition, aneurysm stents are also known for example, which are used primarily to seal the aneurysm.

Stents have a peripheral wall of sufficient supporting strength to hold open the constricted vessel to the desired extent and also a tubular main body, through which the flow of blood continues unimpeded. The peripheral wall is generally formed by a mesh-like supporting structure, which allows the stent to be inserted in a compressed state with small outer diameter as far as the narrowed point to be treated of the respective vessel, where the stent can then be expanded with the aid of a balloon catheter for example until the vessel has the desired, increased inner diameter. Alternatively, shape-memory materials such as Nitinol have the ability to self-expand if there is no restoring force that holds the implant at a small diameter. The restoring force is generally exerted onto the material by means of a protective tube.

In all areas of the medical device accessible from the surrounding environment, specific demands are placed on the material used. Basic preconditions for the use of a material as implant material that comes into contact with the bodily environment when used as intended include its compatibility with the body (biocompatibility). Biocompatibility is understood to mean the ability of a material to induce a suitable tissue response in a specific application. This includes an adaptation of the chemical, physical, biological and morphological surface properties of an implant to the receiver tissue with the objective of a clinically desired interaction. The biocompatibility of the implant material is also dependent on the progression over time of the response of the biosystem into which the material has been implanted or which is in contact at least temporarily with the material. Relatively short-term irritation and inflammation thus occur and may lead to tissue changes. Biological systems therefore respond differently according to the properties of the material. The materials can be divided into bioactive, bioinert and degradable/resorbable materials in accordance with the response of the biosystem.

Materials that are suitable for medical use comprise polymers, metal materials and ceramic materials (for example as a coating). Biocompatible metals and metal alloys for permanent implants or other medical devices for intracorporeal use include for example stainless steels, cobalt alloys, pure titanium and titanium alloys and gold alloys. In the field of biocorrodible implants, in particular stents, the use of magnesium or pure iron as well as biocorrodible master alloys of the elements magnesium, iron, zinc, molybdenum and tungsten is recommended.

Apart from implants, medical devices are also used for diagnosis or treatment in the body. For example, percutaneous transluminal angioplasty (PTA) is a method for expanding or reopening constricted or closed blood vessels by means of balloon dilation. To this end, a balloon catheter in particular is placed into the stenosis via a guide wire and guide catheter and is inflated by means of pressure, whereby the stenosis is remedied. Drug-coated/drug-releasing balloon catheters are a development of conventional balloon catheters. The balloon surface is coated in this case with a drug (for example the cytostatic drug Paclitaxel), which is applied at the point of the vessel constriction. The drug is intended to prevent a vessel-constricting overgrowth of the expanded location.

The medical device according to the invention for intracorporeal use is characterized in that it is formed at least in part from aerographite. In other words, parts (for example a main body of the device) or the entire medical device contains aerographite or consists of aerographite.

The medical device may in particular be an implant or a balloon catheter. Aerographite is therefore used for the first time as a material in medical engineering. The medical device preferably comprises a coating that contains aerographite or consists of aerographite.

Aerographite is a new, ultra-lightweight carbon material. A detailed description of the material and of a way for producing it are described in M. Mecklenburg, A. Schuchardt, Y. K. Mishra, S. Kaps, R. Adelung, A. Lotnyk, L. Kienle, K. Schulte “Aerographite: Ultra Lightweight, Flexible Nanowall, Carbon Microtube Material with Outstanding Mechanical Performance” Advanced Materials 2012, 24, 3486-3490.

Aerographite has a microporous structure, good mechanical properties and is biocompatible, whereby it is outstandingly suitable as a coating of medical devices for intracorporeal use, in particular for implants or balloon catheters. The aerographite is preferably loaded with an active ingredient (drug). Aerographite can be loaded with any active ingredients via its microporous structure. Due to its large surface and electrical conductivity, aerographite is likewise suitable as a material for electrodes or for coating of electrodes, in particular electrodes of cardiac pacemakers or defibrillators.

is Aerographite is produced with use of a zinc oxide sacrificial template. The structure consisting of zinc oxide (the sacrificial template) is gassed for this purpose with a carbonaceous gas, for example toluene. A carbon nanostructure is thus created around the sacrificial template. The zinc oxide is then reduced by means of hydrogen and degasified. The structure produced is flushed with argon and cooled. The properties of the material can be further varied via process parameters, such as gas flow (carbonaceous gas, hydrogen), temperature (gassing, reduction, cooling) and the duration of the different process steps. In particular, aerographites of different density can thus be produced.

In accordance with a preferred embodiment, the aerographite contains zinc oxide. The aerographite containing zinc oxide can be produced for example by just incomplete reduction and degasification of the zinc oxide used as a sacrificial template. Zinc oxide has an anti-inflammatory effect and thus assists the healing process after intracorporeal treatment.

The medical device is an implant in particular. Within the context of the invention, implants are devices introduced into the body via a surgical procedure and include fastening elements for bones, for example screws, plates or pins, surgical suture material, intestinal clamps, vessel clips, prostheses in the region of hard and soft tissue, and electrodes. The implant consists at least in parts of aerographite. In particular, the implant comprises a coating that contains aerographite or consists of aerographite. The implant is preferably a stent or an implantable electrode, in particular for cardiac pacemakers or defibrillators.

In accordance with one aspect of the invention, a medical implant, in particular a stent, is therefore coated with an aerographite. Here, a sacrificial template (as described above) is preferably sintered around a stent having a suitable melting point (for example L605 with a melting point of 1,300-1,400 ° C.). As described above, a carbonaceous gas is injected and the sacrificial template is removed reductively. Here, the production process can be controlled for example via parameters such as shape and density of the sacrificial template, temperature, and gas flow. The implant thus produced, in particular the stent, finds itself after this process in a foam/sponge sleeve formed from aerographite, which can be loaded with any, in particular hydrophobic and medically effective, substances (active ingredients). In the case of a stent, the loading process can be implemented here before or after the process known as “crimping”, that is to say the application of the stent onto a balloon of a balloon catheter for introduction of the stent into a bodily vessel.

The dense, yet porous sleeve made of aerographite thus produced is suitable for closing ruptures (cracks) in bodily vessels due to its hydrophobic properties. This effect can also be boosted by the loading of the aerographite coating with a suitable active ingredient. The implant, in particular the stent, with the aerographite coating may optionally also be supplied to the user, in particular the doctor, without any loading with active ingredient. The loading process can be performed directly at the treatment location by the user by means of a simple dipping method (“dipcoating”). The user can thus select the active ingredient and dosage himself.

In accordance with another aspect of the invention, the implant, in particular the stent, itself is produced from aerographite. Here, the sacrificial template is preferably brought into the shape of the implant and the gas exchange process (gassing, reduction) is carried out as described beforehand.

In particular, a stent is produced from aerographite as described beforehand. A stent of this type is sufficiently mechanically stable to support a bodily vessel. This is true in particular in the case of treatment of a stenosis in a bodily vessel, where the bodily vessel is expanded by means of a balloon and a stent is introduced in order to maintain the expansion of the bodily vessel. Aerographite can be heavily compressed and then readopts the original shape. A stent of this type made of aerographite can therefore be self-expanding.

In the case of a stent or another implant formed from aerographite, there is no need for an additional coating for loading with a medical active ingredient. Aerographite, as already described, is highly porous, and therefore the implant, in particular the stent, can itself be loaded directly with the active ingredient. The loading process here can take place similarly to the previously described loading of a coating formed from aerographite, including the loading carried out by the user.

The stent formed from aerographite in accordance with this embodiment of the invention may have practically any design known in the prior art. In particular, the stent can be formed as simple tubes due to the porous structure of the aerographite.

The porous structure of the aerographite here optimally promotes the ingrowth of the implant, in particular of the stent, into the surrounding bodily tissue, in particular into the surrounding bodily vessel.

In accordance with a further aspect of the invention, the implant is an electrode, in particular an electrode of a cardiac pacemaker or defibrillator. For this purpose, the electrode can be coated with aerographite or produced from aerographite, as detailed beforehand. An electrode according to this aspect of the invention grows more quickly into the surrounding bodily tissue and exhibits an improved pulse transmission as a result of the larger, fractal surface of the aerographite.

In accordance with a further aspect of the invention, the medical device is a balloon catheter, which is used for example for the treatment of a stenosis in a bodily vessel by expansion of the bodily vessel. In particular, a balloon of the balloon catheter is coated with aerographite, that is to say the balloon comprises a coating that contains aerographite or consists of aerographite. To this end, a tube made of zinc oxide with an inner diameter that corresponds approximately to the diameter of the balloon in its folded state is used as a sacrificial template. The folded state of a balloon is understood to mean the state of the balloon that the balloon has during insertion/introduction into the bodily vessel. An aerographite tube is produced form the sacrificial template tubes, similarly to the previously described methods. This aerographite tube is then drawn over the balloon in its folded state and is fastened in a suitable manner (for example by adhesive bonding). The aerographite coating of the balloon thus produced can then be loaded with a suitable active ingredient in the liquid phase, as already detailed. Alternatively, the balloon can be surrounded by a sleeve that contains aerographite or consists of aerographite and is suitably fixed on the balloon catheter.

When treating the stenosis in the bodily vessel by dilation/expansion of the balloon, the aerographite is compressed by the balloon on the one hand and by the bodily vessel on the other hand, whereby, in the case of an active ingredient loading, the active ingredient is released from the aerographite. The active ingredient can thus be introduced directly and selectively into the bodily vessel when treating a stenosis. After expansion/dilation of the balloon, the aerographite returns into its original shape and can be removed with the balloon catheter from the bodily vessel.

The present invention in particular has the following advantages: aerographite can be loaded subsequently with any medically effective substance, which enables a very versatile adaptation of the medical device to the respective requirements in situ. For example, the user can vary the dosage and active ingredient selection. Implants, in particular stents, can be produced more easily and the properties thereof can be improved.

A further aspect of the invention lies in the new use of aerographite in medical engineering, specifically in medical devices for intracorporeal use.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A medical device for intracorporeal use, characterized in that the medical device is formed at least in part from aerographite.

2. The medical device as claimed in claim 1, wherein the medical device comprises a coating that contains aerographite or consists of aerographite.

3. The medical device as claimed in claim 1, wherein the medical device is an implant.

4. The medical device as claimed in claim 3, wherein the implant is a stent.

5. The medical device as claimed in claim 2, wherein the implant is an electrode, in particular a cardiac pacemaker or defibrillator.

6. The medical device as claimed in claim 1, wherein the medical device is a balloon catheter.

7. The medical device as claimed in claim 6, wherein a balloon of the balloon catheter comprises a coating or is surrounded by a sleeve that contains aerographite or consists of aerographite.

8. The medical device as claimed in claim 1, wherein the aerographite is loaded with an active ingredient.

9. The medical device as claimed in claim 1, wherein the aerographite contains zinc oxide.

10. Use of aerographite for producing a medical device for intracorporeal use comprising forming the medical device for intracorporeal use at least in part from aerographite.
